(19) United States
(12) Patent Application Publication
(10) Pub. No.: US 2004/0042992 A1
(43) Pub. Date: Mar. 4, 2004

(54) Makeup removal compositions and method of use

(75) Inventors: Carolyn Romaine, Somerset, NJ (US); Carmen Castillo Bucci, Englewood, NJ (US); Eileen Kim, Denville, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/463,679

(22) Filed: Jun. 18, 2003

Related U.S. Application Data
(60) Provisional application No. 60/398,568, filed on Jul. 26, 2002.

(51) Int. Cl. A61K 7/06; A61K 7/11

(52) U.S. Cl. 424/70.13

(57) ABSTRACT

A composition containing an alkylpolysaccharide, a polyethylene glycol ether of a fatty alcohol, and an oil, useful for makeup removal. Related methods of use and articles of manufacture.

### REFERENCE TO PRIOR APPLICATIONS

This application claims priority to U.S. provisional application No. 60/398,568 filed Jul. 26, 2002, incorporated herein by reference.

### FIELD OF THE INVENTION

The present invention relates to compositions which can be used for removing make up compositions. The invention compositions are efficacious and preferably non stinging to the eye. Methods of use of the invention compositions are also included, as are articles of manufacture incorporating the invention compositions.

### BACKGROUND OF THE INVENTION

Compositions for removal of make up, and especially of a waterproof make up, are known in the art. Mineral oil or isohexadecane are efficacious in removing make-up. However, they tend to be irritating to the eye. Compositions in the form of an emulsion where the mineral oil or isohexadecane is emulsified in the presence of mild surfactants suffer from the drawback of being less efficient, and of clouding the vision temporarily when they spread into the eye. The choice of suitable surfactant needs to balance the lathering characteristics with the mild characteristics. Improving the foam with lather boosters takes away from the mildness. The right blend of surfactants is not easy to attain.

Other ingredients also cause irritation. Preservatives can be irritating to the eye.

Thus there is a need for a composition which is mild, non-irritating, preserved against microbial spoilage, easy to apply and efficacious, for removing make up.

### DETAILED DESCRIPTION OF THE INVENTION

One subject of the present invention is a composition suitable for removing make up preferably without irritation, and its use in this regard. Preferably, the composition is physiologically acceptable. According to one aspect of the invention, there is provided a composition comprising:

a) at least one alkylpolysaccharide;

b) at least one polyethylene glycol ether of a fatty alcohol; and

c) at least one oil.

Alkylpolysaccharide

The compositions of the invention preferably comprise an alkylpolysaccharide having the following general formula (I):

RO(CₓH₂ₓO)ₙZ_{y}

where

Z is a moiety derived from, or of, a reducing saccharide containing 5 or 6 carbon atoms,

R is C₆ to C₃₀ alkyl or alkenyl,

x is 2 or 3,

n is from 1 to 10,

y is from 1 to 10, including all values and subranges therebetween, such as 1.5 to 4.

The alkylpolysaccharide may be present at concentrations ranging from 0.5% to 15%.

Alkylpolysaccharides (also known as alkyl polyglycosides), which can be used in the cosmetic compositions according to the invention, include those commercially available, such as APG®, Glucoponυ, or Plantaren™ surfactants from Cognis Corporation, Ambler, Pa., 19002. Examples of such surfactants include but are not limited to:

APG® 225—an alkylpolyglycoside in which the alkyl group contains 8 to 10 carbon atoms.

APG® 425—an alkyl polyglycoside in which the alkyl group contains 8 to 16 carbon atoms.

APG® 625—an alkyl polyglycoside in which the alkyl groups contains 12 to 16 carbon atoms.

APG® 300—an alkyl polyglycoside substantially the same as the 325 product above but having a different average degree of polymerization.

Glucopon™ 600—an alkylpolyglycoside substantially the same as the 625 product above but having a different average degree of polymerization.

Plantaren™ 2000—a C₈₋₁₆ alkyl polyglycoside having an average degree of polymerization of 1.4.

Plantaren™ 1300—a C₁₂₋₁₆ alkyl polyglycoside having an average degree of polymerization of 1.6.

Plantaren™ 1200—a C1₂₋₁₆ alkyl polyglycoside having an average degree of polymerization of 1.4.

Other non limiting examples include alkyl polyglycoside surfactant compositions such as those sold as Triton, Oramix, or Montanov from SEPPIC, AG from Kao, Atlas G-73500 from Uniqema, Marlosan 240 from Condea Chemie or Desulf GOS-P-60WCG from DeForest Entreprises.

Polyethylene Glycol Ether of a Fatty Alcohol

Suitable polyethylene glycol ethers of fatty alcohols include polyethyleneglycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, olive oil and corn oil, These polyethylene glycol ether of fatty alcohols surfactants may be used at concentrations ranging from 0.01% to 25%, and preferably are surfactants.

Oil

Suitable oils include those commonly used in make up removers. They may consist of a mixture of oils, such as mineral, vegetable or synthetic oils or alternatively silicone oils. Among mineral oils which can constitute the oily phase, there may be mentioned, in particular, liquid paraffin or mineral oil and higher aliphatic hydrocarbons such as, for example, isohexadecane. Among vegetable oils, jojoba oil, meadowfoam seed oil, apricot kernel oil as well as safflower oil. Among silicone oils, cyclopentadimethylsiloxane sold under the name “Volatile Silicone 7158” by the company Union Carbide or DM-fluid 0.6 cs, a linear volatile silicone sold by Shin-Etsu or polydimethylsiloxanes sold as DC200 by Dow Corning. Among synthetic products, esters such as alkylpalmitates in which the alkyl radical has from 2 to 10 carbon atoms, such as isopropyl palmitate or 2-ethylhexyl palmitate, and alkyl adipates in which the alkyl radical has from 2 to 10 carbon atoms, such as bis(2-ethylhexyl) adipate, or esters such as isononyl isononanoate or derivatives such as meadowfoamate. These oils may be used in amounts ranging from 1 to 99.5%.

The compositions of the invention may, in addition, contain adjuvants such as gelling agents, hydrophilic or lipophilic conventional thickening agents; hydrophilic or lipophilic active agents; preservatives, antioxidants; perfumes; emulsifiers; moisturizing agents; chelating agents, sequestering agents, vitamins; emollients; sequestrants; polymers; fillers; anti-free radical agents; ceramides; coloring matter; antioxidants, conditioning agents, preservatives, humectants, proteins, polypeptides, amino acids and derivatives thereof, buffering agents, viscosity control agents, plant extracts or botanicals.

The composition may be applied directly on the area where the make-up is to be removed. It may also be applied on a substrate such as cotton pad, or a woven or non-woven fabric such as a towelette. The presence of a colorant in the composition allows the user to spread the composition more evenly on said pad or support. The composition may be rinsed with water and may also be left on the skin without rinsing. The compositions of the invention are useful in removing make up generally, including both waterproof and transfer-resistant make up.

### EXAMPLES

The following examples are representative of the invention:

Example 1.

| Phase | INCI Name | % w/w |
|---|---|---|
| A1 | Water | 68.089 |
| | Emollient | 0.020 |
| | Buffers | 0.500 |
| | Water soluble preservatives | 0.050 |
| | Allantoin | 0.050 |
| A2 | Humectant | 1.000 |
| | Thickener | 0.200 |
| B | Cocoglucoside | 0.955 |
| | Cetearyl Alcohol | 1.775 |
| | PEG-7 Glycerol Cocoate | 0.910 |
| | Oil soluble Vitamin | 0.100 |
| | Isohexadecane | 18.000 |
| C | Oil Soluble preservative | 0.250 |
| D | Cucumber Extract | 3.000 |
| | Botanicals | 1.101 |
| | Conditioning agent | 4.000 |
| | Total | 100.000 |

This composition was effective in removing make-up and exhibited mild characteristics.

Procedure: In the main beaker add all Phase A1 ingredients with mixing. Heat to 75-80° C. Maintain temperature while homogenizing with Greerco® homogenizer.

In side beaker, prewet the thickening agent in the humectant with spatula until uniform. Add to Phase A1 with continuous mixing and maintain temperature.

In side beaker, add ingredients of phase B. Heat to 78-83° C. Begin agitation with magnetic stir bar. Mix until all ingredients are dissolved and homogeneous.

Make an emulsion by pouring phase B slowly into Phase A, mill for 15 minutes and check under microscope for small particles and under polarized light for the absence of crystals.

Cool to 35-40° C. and add phase C ingredient with mixing until fully dispersed. Add Phase D while mixing. Mix until uniform. Cool batch to 30° C.

### Example 2

| Phase | INCI Name | % w/w |
|---|---|---|
| A1 | Water | 74.530 |
| | Buffers | 0.500 |
| | Water soluble preservatives | 0.650 |
| | Allantoin | 0.050 |
| A2 | Humectant | 1.000 |
| | Thickener | 0.500 |
| | Cucumber Extract | 0.500 |
| B | Cocoglucoside | 0.955 |
| | Cetearyl Alcohol | 1.775 |
| | PEG-7 Glycerol Cocoate | 0.910 |
| | Oil soluble Vitamin | 0.230 |
| | Apricot Kernel Oil | 18.000 |
| D | Coloring agent in Oil | 0.400 |
| | Total | 100.000 |

This composition was effective in removing make-up and exhibited mild characteristics.

Procedure: In the main beaker add all Phase Al ingredients with mixing. Heat to 55-60° C. Maintain temperature while homogenizing with Greerco® homogenizer.

In side beaker, prewet the thickening agent in the humectant with spatula until uniform. Then add Cucumber Extract. Mix until blended. Add to Phase A1 with continuous mixing and maintain temperature.

In side beaker, add ingredients of phase B. Heat to 78-83° C. Begin agitation with magnetic stir bar. Mix until all ingredients are dissolved and homogeneous.

Make an emulsion by pouring phase B slowly into Phase A, mill for 15 minutes and check under microscope for small particles and under polarized light for the absence of crystals.

Cool to 35-40° C. and add phase C ingredient with mixing until fully dispersed. Add Phase D while mixing. Mix until uniform. Cool batch to 30° C.

### Example 3

| Phase | INCI Name | % w/w |
|---|---|---|
| A1 | Water | 46.999 |
| | Emollient | 0.020 |
| | Buffers | 0.500 |
| | Water soluble preservatives | 0.700 |
| | Allantoin | 0.050 |
| | Cucumber extract | 0.260 |
| | Botanicals | 0.501 |
| | Conditioning agent | 0.250 |
| A2 | Humectant | 1.000 |
| | Thickener | 0.500 |
| B | Cocoglucoside | 2.153 |
| | Cetearyl Alcohol | 3.997 |
| | PEG-7 Glycerol Cocoate | 2.050 |
| | Oil soluble Vitamin | 0.230 |
| | Meadowfoam seed oil | 40.540 |
| C | Oil Soluble preservative | 0.250 |
| | Total | 100.000 |

This composition was effective in removing make-up and exhibited mild characteristics.

Procedure: In the main beaker add all Phase A1 ingredients with mixing. Heat to 75-80° C. Maintain temperature while homogenizing with Greerco homogenizer.

In side beaker, prewet the thickening agent in the humectant with spatula until uniform. Add to Phase A1 with continuous mixing and maintain temperature.

In side beaker, add ingredients of phase B. Heat to 78-83° C. Begin agitation with magnetic stir bar. Mix until all ingredients are dissolved and homogeneous.

Make an emulsion by pouring phase B slowly into Phase A, mill for 15 minutes and check under microscope for small particles and under polarized light for the absence of crystals.

Cool to 35-40° C. and add phase C ingredient with mixing until fully dispersed. Add Phase D while mixing. Mix until uniform. Cool batch to 30° C.

### Example 4

| Phase | INCI Name | % w/w |
|---|---|---|
| A1 | Water | 74.38 |
| | Buffers | 0.500 |
| | Water soluble preservatives | 0.800 |
| | Allantoin | 0.050 |
| A2 | Humectant | 1.000 |
| | Thickener | 0.500 |
| | Cucumber Extract | 0.500 |
| B | Cocoglucoside | 0.955 |
| | Cetearyl Alcohol | 1.775 |
| | PEG-7 Glycerol Cocoate | 0.910 |
| | Oil soluble Vitamin | 0.230 |
| | Isononyl Isononanoate | 18.000 |
| C | Coloring Agent in Oil | 0.400 |
| | Total | 100.000 |

This composition was effective in removing make-up and exhibited mild characteristics.

Procedure: In the main beaker add all Phase A1 ingredients with mixing. Heat to 75-80° C. Maintain temperature while homogenizing with Greerco® homogenizer.

In side beaker, prewet the thickening agent in the humectant with spatula until uniform. Add Cucumber Extract. Mix until blended. Add to Phase A1 with continuous mixing and maintain temperature.

In side beaker, add ingredients of phase B. Heat to 78-83° C. Begin agitation with magnetic stir bar. Mix until all ingredients are dissolved and homogeneous.

Make an emulsion by pouring phase B slowly into Phase A, mill for 15 minutes and check under microscope for small particles and under polarized light for the absence of crystals.

Cool to 35-40° C. and add phase C ingredient with mixing until fully dispersed. Add Phase D while mixing. Mix until uniform. Cool batch to 30° C.

### Example 5

| INCI Name | % w/w |
|---|---|
| Cocoglucoside | 1.0 |
| PEG-7 Glyceryl Cocoate | 5.0 |
| Apricot Oil | 96.0 |
| Total | 100.0 |

The composition is effective in removing make-up and is mild.

Procedure: Add ingredients into beaker. Heat to 70-75° C. while mixing with propeller type blade. Mix until homogeneous. Mix and cool to 25° C.

All references mentioned herein, including all texts, patents, applications, articles, etc., are incorporated herein by reference, as is all product information, brochures, etc of commercially available materials mentioned. Where ranges or limits are given all values and subranges therewithin are included as if specifically written out. Where % s are given they are percent by weight based on total weight unless otherwise specified.

What is claimed is:

1. A composition comprising at least one alkylpolysaccharide, at least one polyethylene glycol ether of a fatty alcohol, and at least one oil.

2. A composition according to claim 1, comprising 0.5% to 15% by wt. alkylpolysaccharide, 0.01% to 25% by wt. polyethylene glycol ether of a fatty alcohol, and 1 to 99.5% by wt. oil, all based on total weight of the composition.

3. A composition according to claim 2, comprising an alkylpolysaccharide of the general formula RO(CₓH₂ₓO)ₙZ_{y}
where
Z is a moiety of a reducing saccharide containing 5 or 6 carbon atoms,
R is C₆ to C₃₀ alkyl or alkenyl,
x is 2 or 3,
n is from 1 to 10, and
y is from 1 to 10.

4. A composition according to claim 1, further comprising a colorant.

5. A method, comprising applying the composition of claim 1 to skin having makeup thereon.

6. A method, comprising applying the composition of claim 2 to skin having makeup thereon.

7. A method, comprising applying the composition of claim 3 to skin having makeup thereon.

8. A method, comprising applying the composition of claim 4 to skin having makeup thereon.

9. A method, comprising applying the composition of claim 1 to a substrate and then applying the composition/substrate combination to the skin.

10. The method of claim 9, where the substrate is a cotton pad or a towelette.

11. A method, comprising applying the composition of claim 2 to a substrate and then applying the composition/substrate combination to the skin.

12. The method of claim 11, where the substrate is a cotton pad or a towelette.

13. A method, comprising applying the composition of claim 3 to a substrate and then applying the composition/substrate combination to the skin.

14. The method of claim 13, where the substrate is a cotton pad or a towelette.

15. A method, comprising applying the composition of claim 4 to a substrate and then applying the composition/substrate combination to the skin.

16. The method of claim 15, where the substrate is a cotton pad or a towelette.

17. An article of manufacture comprising a substrate and, in contact with said substrate, the composition of claim 1.

18. An article of manufacture comprising a substrate and, in contact with said substrate, the composition of claim 2.

19. An article of manufacture comprising a substrate and, in contact with said substrate, the composition of claim 3.

20. An article of manufacture comprising a substrate and, in contact with said substrate, the composition of claim 4.
